(19) United States
(12) Patent Application Publication
(10) Pub. No.: US 2003/0158160 A1
(43) Pub. Date: Aug. 21, 2003

(54) Pharmaceutical combination of an anti-androgen and tamoxifen for providing an anti-androgenic effect and aromatase inhibition

(76) Inventors: Barrington Furr, Cheshire (GB); Ian Cockshott, Cheshire (GB)

Correspondence Address: ROPES & GRAY LLP, ONE INTERNATIONAL PLACE, BOSTON, MA, 02110-2624, US

(21) Appl. No.: 10/311,420

(22) PCT Filed: Jul. 4, 2001
(86) PCT No.: PCT/SE01/01548

(30) Foreign Application Priority Data
Jul. 5, 2000 (GB) 0016428.5
Aug. 24, 2000 (GB) 0020767.0

(51) Int. Cl. A61K 31/56; A61K 31/137

(52) U.S. Cl. 514/171; 514/651

(57) ABSTRACT

The present invention relates to a pharmaceutical product, daily dose or dose regimen comprising an anti-androgen and tamoxifen, wherein the anti-androgen is selected from flutamide, nilutamide, chlormadinone acetate and cyproterone or pharmaceutically acceptable salt or solvate thereof. The invention also relates to a method of providing an anti-androgenic effect and anti-oestrogenic effect in a patient, wherein the anti-oestrogenic effect is provided substantially without causing an additional increase in the levels of circulating androgens.

### PHARMACEUTICAL COMBINATION

The present invention relates to a pharmaceutical product, daily dose or dose regimen comprising an anti-androgen and tamoxifen, wherein the anti-androgen is selected from flutamide, nilutamide, chlormadinone acetate and cyproterone. The invention also relates to a method of providing an anti-androgenic effect and an anti-oestrogenic effect in a patient, wherein the anti-oestrogenic effect is provided substantially without causing an additional increase in the levels of circulating androgens. Furthermore, the invention relates to the use of an anti-androgen and tamoxifen in the manufacture of a pharmaceutical product for this purpose, wherein the anti-androgen is selected from flutamide, nilutamide, chlormadinone acetate and cyproterone.

### BACKGROUND TO THE INVENTION

Tamoxifen, an anti-oestrogen, is known by the AstraZeneca trade name NOLVADEX Tamoxifen is the trans isomer of 1-(p-beta-dimethyaminoethoxyphenyl)-1,2-diphenylbut-1-ene, which is disclosed in U.S. Pat. No. 4,536,516. An alternative name is (Z)-2-[p-(1, 2-diphenylbut-1-enyl)phenoxy]ethyldimethylamine. The corresponding structure is shown in formula I:

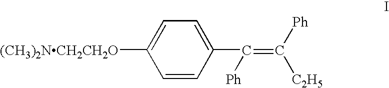

Flutamide, an anti-androgen, is known by the trade name EULEXIN™. Flutamide is also known by the alternative names 2-methyl-N-[4-nitro-3-(trifluoromethyl)phenyl]propanamide; α,α,α-trifluoro-2-methyl-4′-nitro-m-propionotoluidide; and 4′-nitro-3′-trifluoromethylisobutyranilide. Flutamide is disclosed in U.S. Pat. No. 3,847,988. The corresponding structure is shown in formula II:

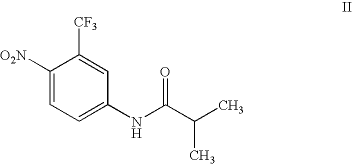

Nilutamide, an anti-androgen, is known by the trade name NILANDRON™. Nilutamide is also known by the alternative names 5,5-dimethyl-3-[4-nitro-3-(trifluoromethyl)phenyl]-2,4-imidazolidinedione; and 1-(3′-trifluoromethyl-4′-nitrophenyl)-4,4-dimethylimidazoline-2,5-dione. Nilutamide is disclosed in U.S. Pat. No. 4,097,578. The corresponding structure is shown in formula III:

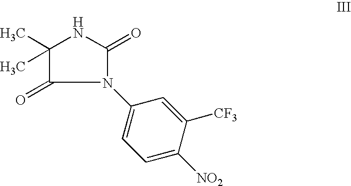

Chlormadinone, in its acetate form, is an anti-androgen. The acetate form is known by the alternative names 17-(acetyloxy)-6-chloropregna-4,6-diene-3,20-dione; 6-chloro-17-hydroxypregna-4,6-diene-3,20-dione acetate; 6-chloro-6-dehydro-17α-hydroxyprogesterone acetate; 6-chloro-6-dehydro-17α-acetoxyprogesterone; and 17α-acetoxy-6-choro-6,7-dehydroprogesterone. Chormadinone is disclosed in U.S. Pat. No. 3,485,852.

Cyproterone is known by the alternative names (1β,2β)-6-chloro-1,2-dihydro-17-hydroxy-3′H-cyclopropa[1,2]pregna-1,4,6-triene-3,20 dione; 6-chloro-17-hydroxy-1α,2α-methylenepregna-4,6-diene-3,20-dione; 6-chloro-6-dehydro-17α-hydroxy-1α,2α-methyleneprogesterone; and 6-chloro-1,2α-methylene-4,6-pregnadien-17α-ol-3,20-dione. Cyproterone is disclosed in U.S. Pat. No. 3,234,093. Cyproterone in its free alcohol and acetate forms is an anti-androgen.

Anti-androgens such as flutamide and nilutamide are used in the treatment of prostate cancer. This is also the case for another anti-androgen, bicalutamide. Such compounds are generally used in combination with an inhibitor of gonadotrophin secretion, for example a luteinising hormone releasing hormone (LHRH) agonist such as goserelin, buserelin, leuprorelin or triptorelin. The properties and usefulness of these anti-androgens have been reviewed, for example in the following documents which are incorporated herein by way of reference:

flutamide R O Neri, J. Drug Develop., 1987, 1 (Suppl.), 5-9 and Urology, 1989, 34 (Suppl. 4),19-21 and United Kingdom Patent Application No. 1360001;

bicalutamide B J A Furr et al., Urology, 1996,47 (Suppl.1A), 13-25, G J C Kolvenbag et al., Urology, 1996, 47 (Suppl. 1A), 70-79 and European Patent Application No. 0100172 as the 8th compound listed in the table in Example 6;

nilutamide M G Harris et al., Drugs and Aging, 1993, 3, 9-25 and United Kingdom Patent Application No.1518444.

It has been observed that administration of flutamide, bicalutamide or nilutamide in single agent therapy to humans causes an increase in the amount of testosterone circulating in the blood. For example, it has been disclosed that administration of bicalutamide leads to an approximate doubling of the basal level of circulating testosterone (G R P Blackledge et al., Urology, 1996, 47 (Suppl. 1A), 44-47). Likewise, it has been disclosed that administration of flutamide causes a 50 to 80% increase in the basal level of circulating testosterone (L Boccon-Gibod et al., J. Urology, 1992, 147, 417A, Abstract 818 and European Urology. 1997, 32, 391-395 and Brufsky et al., Urology, 1997, 49, 913-920). Likewise, administration of nilutamide causes an increase in the basal level of circulating testosterone (A U Decensi et al., J. Urology, 1991, 146, 377-381). It is believed that such increases in the level of testosterone occur when sufficient of the anti-androgen gains access to the CNS and blocks androgen receptors in the hypothalamus. The consequential lack of feedback of androgen causes additional release of LHRH by the hypothalamus which in turn causes release of luteinising hormone (LH) and follicle stimulating hormone (FSH) by the pituitary gland and production of testosterone in the testes. Aromatase enzyme in fat and other tissues converts some of the increased concentration of testosterone to oestradiol, which results in increased concentrations of oestrogen in the blood. Further discussion of this is provided by C Mahler et al, Clinical Pharmacokinetics, 1998, 34(5), pp 405417.

A disadvantageous effect is produced. Namely, the increase in the levels of circulating oestrogen may cause one or more of the side effects of gynaecomastia, breast tenderness, hot flushes, impotence and reduction in libido. A discussion on gynaecomastia can be found in C J Tyrrell, Prostate Cancer and Prostatic Diseases, 1999, 2(4): pp 167-171.

As explained above, the testosterone and LH levels tend to rise. Mahler et al explain that the rising oestrogen levels progressively activate the normal feedback mechanism, and so the rise in LH and testosterone is limited. It is widely accepted in the art that oestrogen levels are important in regulating LH secretion, and by this means testosterone secretion, as invoked by Mahler et al. It is clear from numerous publications that the reduction of the negative feedback effect of oestrogens on the hypothalamic-pituitary axis in men and male animals results in an increase in luteinising hormone (LH) secretion. This in turn drives the testes to produce increased quantities of testosterone. In this respect, reference is made to F H Comnhaire et al, Human Reproduction, 1995, 10 (7), pp 1740-1744, where tamoxifen (an anti-oestrogen) intake in adult men was reported to increase testosterone and LH.

J J Spijkstra et al, J. Clinical Endocrinology and Metabolism, 1988, 66(2), pp 355-360, reports a study of LH secretion in 13 normal men before and after the administration of tamoxifen for a 6 week period. An increase in mean serum testosterone, oestradiol, LH levels, LH pulse frequency and LH pulse amplitude were observed after tamoxifen administration. Similar results were cited in men given the anti-oestrogen clomiphene citrate. Spijkstra et al suggest that the observed result with tamoxifen was due to an inhibition of negative feedback on pituitary oestrogen receptors.

D I Lewis-Jones et al, Andrologia 1987, 19(1): pp 86-90 reports that tamoxifen administration to men elevates the basal serum levels of LH, oestradiol “and particularly testosterone. The marked elevation in serum testosterone levels produced by the administration of tamoxifen may be a more successful method for elevating male hormone levels than the use of other pharmacological agents such as mesterolone”.

L van Bergeijk et al, Horm. Metabol. Res., 1986, pp 558-564, reports that three months' treatment with tamoxifen in normogonadotrophic oligozoospermic men stimulated basal LH, FSH and testosterone levels. They suggested that oestrogens play a role in the negative feedback regulation of gonadotrophin release.

There is, therefore, a prejudice in the art against using an anti-oestrogen to combat the rise in oestrogen levels observed when an anti-androgen is administered to a male. This is because the anti-oestrogen would be expected, in view of the numerous previously reported studies, to produce a substantial additional increase in LH and testosterone, which in turn would be expected to compromise the anti-androgenic effect of the anti-androgen.

There is therefore a need for a treatment that can provide an anti-androgenic effect and combat the rise in oestrogen levels, thereby suppressing a side effect selected from gynaecomastia, breast tenderness, hot flushes, impotence and reduction in libido, without substantially causing an additional increase in the levels of circulating androgens above the levels produced by the anti-androgen alone.

### SUMMARY OF THE INVENTION

The present invention fulfils this need by providing a pharmaceutical product for administration to a patient for providing an anti-androgenic effect and anti-oestrogenic effect in the patient, the product comprising an anti-androgen and tamoxifen or a pharmaceutically acceptable salt or solvate thereof, wherein the anti-androgen is selected from flutamide, nilutamide, chlormadinone acetate and cyproterone or a pharmaceutically acceptable salt or solvate thereof The cyproterone is in its free alcohol or acetate form. Preferably, the anti-androgen and tamoxifen are provided in a ratio of 25 to 1000:0.5 to 100 respectively.

As a result of the present invention, the anti-oestrogenic effect is provided substantially without causing an additional increase in the levels of circulating androgens. By this, we mean that the androgen levels (eg, as indicated by total or free testosterone in blood) in the patient do not substantially increase above the level usually observed when the anti-androgen alone is administered to patients.

The present invention also provides a daily pharmaceutical dose for administration to a patient for providing an anti-androgenic effect and anti-oestrogenic effect in the patient, the dose comprising an anti-androgen and from 0.5 to 100 mg of tamoxifen or a pharmaceutically acceptable salt or solvate thereof, wherein the anti-androgen is selected from flutamide, nilutamide, chlormadinone acetate and cyproterone or a pharmaceutically acceptable salt or solvate thereof.

In addition, the present invention provides a dose regimen for such purpose comprising an anti-androgen and from 0.5 to 100 mg of tamoxifen or a pharmaceutically acceptable salt or solvate thereof for simultaneous or sequential administration to the patient, wherein the anti-androgen is selected from flutamide, nilutamide, chlormadinone acetate and cyproterone or a pharmaceutically acceptable salt or solvate thereof.

Other aspects of the invention relate to the use in the manufacture of a pharmaceutical product of an anti-androgen and tamoxifen or a pharmaceutically acceptable salt or solvate thereof that are simultaneously or sequentially administrable to a patient, for:

(a) providing an anti-androgenic effect and anti-oestrogenic effect in the patient, wherein the anti-oestrogenic effect is provided substantially without causing an additional increase in the levels of circulating androgens; or

(b) providing an anti-androgenic effect in the patient and suppressing increase in the incidence or severity of a side effect selected from gynaecomastia, breast tenderness, hot flushes, impotence and reduction in libido, substantially without causing an additional increase in the levels of circulating androgens;

wherein the anti-androgen is selected from flutamide, nilutamide, chlormadinone acetate and cyproterone or a pharmaceutically acceptable salt or solvate thereof.

By “suppressing increase in the incidence or severity of a side effect”, we mean providing a lower incidence or severity compared with the side effect produced when the anti-androgen is administered alone, or eliminating the side effect.

The present invention further provides a method of providing an anti-androgenic effect in a patient comprising simultaneously or sequentially administering an anti-androgen and tamoxifen or a pharmaceutically acceptable salt or solvate thereof to the patient, wherein the method further provides anti-oestrogenic effect in the patient substantially without causing an additional increase in the levels of circulating androgens, and wherein the anti-androgen is selected from flutamide, nilutamide, chlormadinone acetate and cyproterone or a pharmaceutically acceptable salt or solvate thereof

### DETAILED DESCRIPTION OF THE INVENTION

The present invention provides both an anti-androgenic effect and anti-oestrogenic effect in a patient, wherein the anti-oestrogenic effect is produced substantially without causing an additional increase in the levels of circulating androgens. This is achieved by administering to the patient a product comprising an anti-androgen and tamoxifen or a pharmaceutically acceptable salt or solvate. thereof, wherein the anti-androgen is selected from flutamide, nilutamide, chlormadinone acetate and cyproterone or a pharmaceutically acceptable salt or solvate thereof. Preferably, the anti-androgen and tamoxifen are provided in a ratio respectively of 25 to 1000 (preferably the lower end of the range being 50 or 100; preferably the upper end of the range being 500, 350, 300, 150 or 50; suitable values in the ranges being 750, 375, 150, 125 or 50): 0.5 to 100 (preferably the lower end of the range being 1 or 5; preferably the upper end of the range being 40, 20 or 10; a suitable value in the range being 20). For flutamide, a preferred range is 100 to 1000, and 750 or 375 is a preferred value. For chlormadinone acetate a preferred value is 50. For cyproterone a preferred range is 200 to 300. For nilutamide, a preferred range is 50 to 500, and 300 or 150 is a preferred value. The term “product” is intended to mean either a mixture of the anti-androgen and tamoxifen (eg, provided as a capsule or tablet containing both compounds) or a kit comprising separate amounts of the compounds (eg, a set of tamoxifen tablets and a separate set of tablets of the anti-androgen). The latter product can be used for simultaneous or sequential (ie, temporally spaced) administration of the compounds to the patient, while the pre-mixed compounds are for simultaneous administration. Factors such as the rate of absorption, metabolism and the rate of excretion of each agent will affect their presence at the tumour site. Such factors are routinely considered by, and are well within the ordinary skill of, the clinician when he contemplates the treatment of a medical condition which requires the conjoint administration of two agents in order to obtain a beneficial effect.

The invention contemplates the use of pharmaceutically acceptable salts and solvates of the anti-androgen (for flutamide and nilutamide) and/or tamoxifen. Suitable salts are, for example acid addition salts, such as hydrochloride, hydrobromide, phosphate, acetate, fumarate, maleate, tartarate, citrate, oxalate, methanesulphonate or p-toluenesulphonate, or alkali metal salts such as sodium or potassium salts.

The tamoxifen is included to provide an anti-oestrogenic effect, in that this compound prevents oestrogen activity.

The anti-androgenic effect is useful for treating cancer, for example prostate cancer. Particular examples are advanced prostate cancer and early prostate cancer. The anti-androgenic effect may be useful for prophylaxis, in order to reduce the risk of prostate cancer occurrence in patients. This could be especially useful in men genetically pre-disposed to prostate cancer. Conventional methods are available to classify patients according to their risk of contracting prostate cancer, for example by assessment of family history and measurements over time of particular blood proteins such as prostate specific antigen (PSA). Other uses for the anti-androgenic effect are the treatment of a non-malignant disease of the prostate gland (eg, benign prostatic hyperplasia or hypertrophy) and acne.

The anti-oestrogenic effect is useful for suppressing increase in the incidence or severity of a side effect selected from gynaecomastia, breast tenderness, hot flushes, impotence, reduction in libido, nausea, vomiting, fatigue and diarrhoea. Such side effects have been observed with monotherapy use of anti-androgens. Preferably, the side effect is one or both of gynaecomastia and breast tenderness.

A suitable dose regimen or daily pharmaceutical dose comprises the anti-androgen and from 0.5 to 100 mg of tamoxifen or a pharmaceutically acceptable salt or solvate thereof.

Preferably, for tamoxifen the lower end of the range is 1 or 5 mg; preferably the upper end of the range is 40, 20 or 10 mg; a suitable value in the range being 20 mg. The dose or the regimen preferably comprises from 25 to 1000 mg of the anti-androgen or a pharmaceutically acceptable salt or solvate thereof Preferably the lower end of the range is 50 or 100 mg; preferably the upper end of the range is 350, 300, 150 or 50 mg; suitable values in the ranges are 750, 375, 150, 125 or 50 mg. For flutamide, a preferred range is 100 to 1000 mg, and 750 or 375 mg is a preferred value. For chlormadinone acetate a preferred value is 50 mg. For cyproterone a preferred range is 200 to 300 mg. For nilutamide, a preferred range is 50 to 500 mg, and 300 or 150 mg is a preferred value.

For the regimen, each compound is preferably administered daily. Another possible regime would be dosing of the anti-androgen on alternate days and dosing of the tamoxifen also on (the same or different) alternate days. To this end, the regimen may include administration instructions. Alternatively, a dose of the anti-androgen is administered every 3, 4, 5, 6 or 7 days and the tamoxifen is administered every 3, 4, 5, 6 or 7 days (eg, on the same day as the anti-androgen).

In one embodiment, the regimen or daily dose comprises 3 times 250 mg of flutamide (eg, 250 mg administered every 8 hours) or 3 times 125 mg of flutamide (eg, 125 mg administered every 8 hours).

The patient can be a human male, eg an adult, but the treatment of other mammals (except rats) is also contemplated.

The products, doses and regimens of the invention may be in a form suitable for oral use (for example as tablets, capsules, aqueous or oily suspensions, emulsions or dispersible powders or granules), for topical use (for example as creams, ointments, gels, or aqueous or oily solutions or suspensions; for example for use within a transdermal patch), for parenteral administration (for example as a sterile aqueous or oily solution or suspension for intravenous, subcutaneous, intramuscular or intravascular dosing), or as a suppository for rectal dosing. Preferably the compositions of the invention are in a form suitable for oral use, for example as tablets or capsules.

The products, doses and regimens of the invention may be obtained by conventional procedures using conventional pharmaceutically-acceptable diluents or carriers that are well known in the art.

Suitable pharmaceutically-acceptable diluents or carriers for a tablet formulation include, for example, inert diluents such as lactose, sodium carbonate, calcium phosphate or calcium carbonate, granulating and disintegrating agents such as corn starch or alginic acid; binding agents such as gelatin or starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin or olive oil.

When we mention providing anti-oestrogenic effect without causing an additional increase in the levels of circulating androgens, we mean that the androgen levels (eg, as indicated by total or free testosterone in blood) in the patient do not substantially increase above the maximum level usually observed when the anti-androgen alone is administered to patients. An enabling illustration is provided in the human clinical trial below. While this relates to the use of NOLVADEX™ (tamoxifen) in combination with CASODEX™ (bicalutamide), it is expected that the use of a combination according to the present invention in a similar trial also demonstrates the effect.

Human Clinical Trial

The following clinical trial was performed to determine the effect of the administration of CASODEX™ together with NOLVADEX™ on free testosterone levels in healthy male volunteers over a 6 week period.

Protocol

Key Inclusion Criteria: Male, aged 65 years or above showing no clinically significant abnormalities in routine haematological and biochemical tests and having endocrinology and prostate specific antigen (PSA) results within normal limits.

Key Exclusion Criteria: Previous inclusion in a clinical trial using CASODEX™; concurrent treatment with any drugs with the exception of paracetomol; history or presence of any testicular abnormality; history or presence of gastrointestinal, hepatic or renal disease, or other condition known to interfere with the absorption, distribution, metabolism or excretion of drugs; a clinically significant illness within 2 weeks of trial commencement; definite or suspected personal or family history of significant adverse drug reactions or any hypersensitivity to CASODEX™ or NOLVADEX™; treatment within the previous 3 months with any drugs known to have a well-defined potential for hepatotoxicity or hepatic interaction.

Dosage: The CASODEX™ was administered daily at a dose of 150 mg and the NOLVADEX™ was administered daily at a dose of 20 mg. All treatments were in tablet form and taken once daily. Daily treatment with CASODEX™ plus NOLVADEX™ was for 6 weeks, this period being selected as the minimum time to attain steady-state plasma concentrations for the drugs. Another set of volunteers were administered CASODEX™ alone at a daily dose of 150 mg for 4 weeks. All treatments were in tablet form and taken once daily.

Key Assessment: Free testosterone concentrations were measured during the course of the trial.

Results

Summaries of the free testosterone concentrations over the treatment periods are presented in Table 1 (for CASODEX™ in combination with NOLVADEX™) and Table 2 for CASODEX™ alone.

TABLE 1
Free testosterone concentrations following treatment with
CASODEX ™ plus NOLVADEX ™

| Parameter | Day 1 | Day 8 | Day 22 | Day 43 | Follow-up |
|---|---|---|---|---|---|
| Testosterone | | | | | |
| (nmol/l) | | | | | |
| n | 7 | 7 | 7 | 7 | 7 |
| gmean | 0.045 | 0.059 | 0.077 | 0.063 | 0.056 |
| CV | 11.970 | 18.628 | 34.582 | 34.303 | 22.686 |
| Minimum | 0.04-0.05 | 0.05-0.08 | 0.04-0.10 | 0.04-0.09 | 0.04-0.07 |
| Ratio to | — | 1.30 | 1.69 | 1.38 | 1.23 |
| Day 1 | | | | | |

CV = Coefficient of variation gmean = Geometric mean n = Number of observations

Day 1 samples were drawn before dosing, and therefore act as a baseline measurement.

No volunteers in the CASODEX™ plus NOLVADEX™ group experienced gynaecomastia.

TABLE 2
Free testosterone concentrations following treatment with
CASODEX ™ alone

| Parameter | Day 1 | Day 29 |
|---|---|---|
| Testosterone (nmol/l) | | |
| n | 7 | 7 |
| gmean | 0.048 | 0.076 |
| CV | 30.415 | 26.219 |
| Minimum | 0.03-0.07 | 0.00-0.12 |
| Ratio to Day 1 | — | 1.58 |

CV = Coefficient of variation gmean = Geometric mean n = Number of observations

Day 1 samples were drawn before dosing, and therefore act as a baseline measurement.

Conclusion

When CASODEX™ alone was administered, the mean free testosterone concentration increased 58% by the end of the treatment period. With continued administration of CASODEX™ beyond the 4 week, this figure would be expected to rise (corresponding to an approximate doubling of the mean total testosterone concentration). In this respect, reference is made to a trial reported by Verhelst, J et al (“Endocrine profiles during administration of the new non-steroidal anti-androgen Casodex in prostate cancer”, Verhelst, J et al, Clin. Endocrinol. (Oxf) 1994, Oct., 41(4), pp 525-30), which reported an increase of 57% in the mean free testosterone concentration after 24 weeks of daily administration of 150 mg CASODEX™ alone.

Reference to Table 1 shows that the co-administration of NOLVADEX™ with CASODEX™ produced no additional clinically significant change in the mean concentration of free testosterone. Indeed, by the end of the treatment period the increase in the mean concentration was 38%.

The results therefore support the present invention wherein the tamoxifen does not compromise the anti-androgenic effect, in that contrary to the expectations of the skilled person based on the aforementioned prejudice in the art, the tamoxifen does not cause an additional increase in the levels of androgens beyond the levels expected when anti-androgen alone is used.

1. A pharmaceutical product for administration to a patient for providing an anti-androgenic effect and anti-oestrogenic effect in the patient, the product comprising an anti-androgen and tamoxifen or a pharmaceutically acceptable salt or solvate thereof, wherein the anti-androgen is selected from flutamide, nilutamide, chlormadinone acetate and cyproterone or a pharmaceutically acceptable salt or solvate thereof.

2. The pharmaceutical product of claim 1, wherein the anti-androgen and tamoxifen are provided in a ratio of 25 to 10000.5 to 100 respectively.

3. A daily pharmaceutical dose for administration to a patient for providing an anti-androgenic effect and anti-oestrogenic effect in the patient, the dose comprising an anti-androgen and from 0.5 to 100 mg of tamoxifen or a pharmaceutically acceptable salt or solvate thereof, wherein the anti-androgen is selected from flutamide, nilutamide, chlormadinone acetate and cyproterone or a pharmaceutically acceptable salt or solvate thereof.

4. A dose regimen for providing an anti-androgenic effect and anti-oestrogenic effect in a patient, the regimen comprising an anti-androgen and from 0.5 to 100 mg of tamoxifen or a pharmaceutically acceptable salt or solvate thereof for simultaneous or sequential administration to the patient, wherein the anti-androgen is selected from flutamide, nilutamide, chlormadinone acetate and cyproterone or a pharmaceutically acceptable salt or solvate thereof.

5. The dose of claim 3, or the regimen of claim 4, comprising from 25 to 1000 mg of the anti-androgen.

6. Use in the manufacture of a pharmaceutical product of an anti-androgen and tamoxifen or a pharmaceutically acceptable salt or solvate thereof for simultaneous or sequential administration to a patient, for providing an anti-androgenic effect and anti-oestrogenic effect in the patient, wherein the anti-oestrogenic effect is provided substantially without causing an additional increase in the levels of circulating androgens, and wherein the anti-androgen is selected from flutamide, nilutamide, chlormadinone acetate and cyproterone or a pharmaceutically acceptable salt or solvate thereof.

7. Use in the manufacture of a pharmaceutical product of an anti-androgen and tamoxifen or a pharmaceutically acceptable salt or solvate thereof for simultaneous or sequential administration to a patient, for providing an anti-androgenic effect in the patient and suppressing increase in the incidence or severity of at least one side effect selected from gynaecomastia, breast tenderness, hot flushes, impotence and reduction in libido, substantially without causing an additional increase in the levels of circulating androgens, wherein the anti-androgen is selected from flutamide, nilutamide, chlormadinone acetate and cyproterone or a pharmaceutically acceptable salt or solvate thereof.

8. A method of providing an anti-androgenic effect in a patient comprising simultaneously or sequentially administering an anti-androgen and tamoxifen or a pharmaceutically acceptable salt or solvate thereof to the patient, wherein the method further provides anti-oestrogenic effect in the patient substantially without causing an additional increase in the levels of circulating androgens, and wherein the anti-androgen is selected from flutamide, nilutamide, chlormadinone acetate and cyproterone or a pharmaceutically acceptable salt or solvate thereof.
